(19) United States
(12) Patent Application Publication
(10) Pub. No.: US 2010/0119519 A1
(43) Pub. Date: May 13, 2010

(54) OPHTHALMIC DRUG DELIVERY SYSTEM AND METHOD

(76) Inventor: Gholam A. Peyman, Sun City, AZ (US)

Correspondence Address: THOMPSON HINE L.L.P.;Intellectual Property Group, P.O. BOX 8801, DAYTON, OH, 45401-8801, US

(21) Appl. No.: 12/611,682

(22) Filed: Nov. 3, 2009

Related U.S. Application Data
(60) Provisional application No. 61/114,143, filed on Nov. 13, 2008.

(51) Int. Cl. A61F 2/14; A61K 47/30; A61K 38/13; A61K 31/436; A61K 31/365; A61K 39/395

(52) U.S. Cl. 424/141.1; 623/4.1; 514/772.3; 514/11; 514/291; 514/470

(57) ABSTRACT

An ocular device comprising a delivery body configured for implanting within the capsular bag of a patient's eye, the delivery body containing an ocular therapeutic agent, the delivery body having a permeable exterior surface for delivering the therapeutic agent when implanted in the patient's eye.

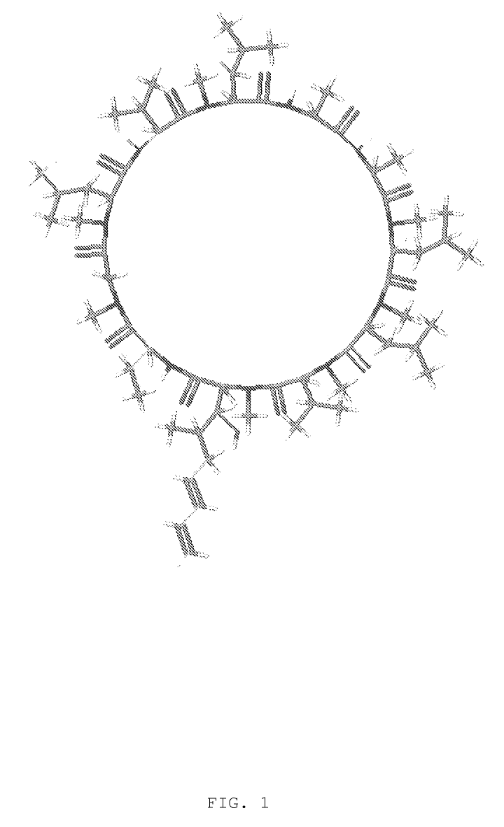

This application claims priority from U.S. application Ser. No. 61/114,143 filed Nov. 13, 2008, the contents of which are expressly incorporated by reference herein in its entirety.

### BRIEF DESCRIPTION OF THE DRAWING

The FIGURE shows a sample drug that can be used in the inventive system and method.

Known methods of drug delivery to the eye have drawbacks, as the following illustrations demonstrate. Topical drug deliver must be repeated many times on a daily basis because of low or slow penetration. Compliance is also a problem. Subconjunctival drug delivery can be painful and has slow drug penetration. Intravitreal drug delivery has a short duration, typically of 2 to 30 days, so additional intervention and/or repeated injections are needed. The possibility of potential infections and retinal injury are also problems. Scleral implants and trans-scleral implants have not been attempted or tested. The implanted devices usually are made of polymers; there is usually slow intraocular penetration when polymers are injected into the eye. The vitreous usually requires additional intervention with attendant potential complications, such as infection, retinal injury, etc.

Method of intraocular delivery of various therapeutic agents and methods are disclosed in Peyman et al., Retina, The Journal of Retinal and Vitreous Diseases 29 (2009) 875-912, which is expressly incorporated by reference in its entirety.

The disclosed system and method uses the capsular bag, obtained during or after cataract extraction, as a polymeric slow release drug delivery system and method. It is used for drug delivery and for simultaneous support for the lens capsule.

The inventive system is used during or after intra-ocular surgery for cataract extraction in the same session. After an opening in the anterior chamber is made, a circular area of the anterior capsule is removed to extract the lens cortex and nucleus.

In one embodiment, the system and method is used post-surgically to prevent or to treat inflammation. After surgery, most if not all eyes have some inflammation for which treatment is administered. For example, all patients who have diabetic retinopathy have post-surgical ocular inflammation. All patients who have a previous history of uveitis have more excessive inflammation.

The device may be of any shape. The following embodiments are illustrative only and are not limiting. In one embodiment, the device is ring shaped. In one embodiment, the device is shaped as an open ring (e.g., doughnut or tire shape). In one embodiment, the device is shaped as a rod, which may be straight or curved. In one embodiment, the device is shaped as a semicircle. In one embodiment, the device contains one ring. In one embodiment, the device contains at least two concentric rings. In one embodiment, the device is shaped as an oval. In one embodiment, the device is C shaped. In one embodiment, the device is shaped as triangle. In one embodiment, the device is shaped as a quadratic. In one embodiment, the device is spring-shaped. In one embodiment, the device is shaped in a zigzag configuration.

In one embodiment, the size of the device ranges from 1 mm in diameter up to about 34 mm in diameter. In one embodiment, the size of the device ranges from 1 mm in diameter up to about 20 mm in diameter. In one embodiment, the thickness of the device may range from about 50 μm to about 3000 μm. In one embodiment, the thickness of the device may range from about 10 μm to about 3000 μm. In one embodiment, the device is made from a polymeric material that is absorbable. In one embodiment, the device is made from a polymeric material that is nonabsorbable, e.g., polylactic acid polyglycolic acid, silicone, acrylic, polycaprolactone, etc. In one embodiment, the device is made as microspheres.

The device is positioned in the lens capsule, e.g., after cataract extraction prior to or after IOL implantation. In one embodiment, it is positioned inside the lens capsule after cataract extraction and acts as a polymeric capsular expander keeping the capsular bag open for intraocular lens (IOL) implantation). In one embodiment, the device is positioned on the haptics of the IOL. In one embodiment, the device is located inside the capsule or under the iris supported by the lens zonules, or it can be sufficiently large to lie in the ciliary sulcus, or ciliary body, or hanging from the zonules in a C-shaped configuration.

For implantation, after removing the lens cortex and nucleus inside the capsule through a capsulotomy, the inventive device is implanted before or after an IOL is implanted. The inventive device is flexible, deformable, and re-moldable. In one embodiment, the inventive device is implanted through a incision one mm or less using an injector, forceps, etc. The incision may be made in the cornea for cataract removal. In one embodiment, the inventive device is implanted in an eye without cataract extraction. In this embodiment the inventive device may be implanted under the iris, e.g., after traumatic anterior segment injury, and lies over the crystalline lens, IOL, and zonules. Implantation may be facilitated by using a visco-elastic material such as healon, methyl cellulose, etc.

Retino-choroidal diseases are aggravated after cataract surgery. Retino-choroidal diseases include, but are not limited to, diabetes, existing prior inflammations such as uveitis, vascular occlusion, wet age related macular degeneration, etc. Patients with these diseases are candidates for the inventive drug delivery system and method. Other indications are prophylactic therapy prior to development of retinal complications, such as inflammation (CME) and infection, and therapy for an existing disease. Other indications are conditions in which any intraocular drug delivery to treat aging processes if cataract surgery is contemplated or after IOL implantation. In latter situation, the inventive device can be implanted in the capsule or over the IOL under the iris Other indications are post-surgical inflammations, post-surgical infections such as after cataract extraction, and any intraocular delivery.

In one embodiment, medication can be coated on a surface and eluted from the surface of the inventive device for delivery, using methods known in the art (e.g., drug-coated stents). In one embodiment, medication can be incorporated in the polymeric material using methods known to one skilled in the art. The following medications can be delivered, alone or in combinations, to treat eyes using the inventive system and method: steroids, non-steroidal anti-inflammatory drugs (NSAIDS), antibiotics, anti-fungals, antioxidants, macrolides including but not limited to cyclosporine, tacrolimis, rapamycin, mycophenolic acid and their analogs, etc. For example, voclosporin (FIG.) is a next generation calcineurin inhibitor, an immunosuppressive compound, developed for the treatment of uveitis, an inflammation of the uvea, the treatment of psoriasis, and for the prevention of organ rejection in renal transplant patients. It can be used with other immunomodulatores, etanercept, infliximab, adalimumab, etc. Other examples include: antibodies (e.g., anti-vascular endothelial growth factor), immunomodulators, antiproliferative agents, gene delivery agents (e.g., to treat damaged neuronal tissue), neuroprotective agents, anti-glaucoma agents (e.g., to treat or prevent increases in intraocular pressure, etc.). In one embodiment, combinations of agents may be provided in a single device or in multiple devices.

The duration of delivery is manipulated so that the agent(s) is released at a quantity needed to achieve therapeutic effect for each agent, if more than one agent is administered, as long as necessary. Duration may be a single dose, may be one day, may be daily for up to 12 months or longer, may be several times a day. In embodiments using a polymer, reimplantation is possible through a small incision once the polymer is absorbed.

Other variations or embodiments will be apparent to a person of ordinary skill in the art from the above description. Thus, the foregoing embodiments are not to be construed as limiting the scope of the claimed invention.

What is claimed is:

1. An ocular device comprising a delivery body configured for implanting within the capsular bag of a patient's eye, the delivery body containing an ocular therapeutic agent, the delivery body having a permeable exterior surface for delivering the therapeutic agent when implanted in the patient's eye.

2. The device of claim 1 made from a synthetic polymer or organic material.

3. The device of claim 1 made from the lens capsule.

4. The device of claim 2 where the therapeutic agent is within the synthetic polymer.

5. The device of claim 2 wherein the therapeutic agent is on a surface of the device.

6. The device of claim 1 wherein the therapeutic agent is selected from the group consisting of steroids, non-steroidal anti-inflammatory drugs (NSAIDS), antibiotics, anti-fungals, antioxidants, macrolides including but not limited to cyclosporine, tacrolimis, rapamycin, mycophenolic acid and their analogs, voclosporin, immunomodulatores, etanercept, infliximab, adalimumab, antibodies, antiproliferative agents, gene delivery agents, neuroprotective agents, anti-glaucoma agents, and combinations thereof.

7. An ocular device comprising a capsular bag extracted from a patient's eye after surgical lens removal, the capsular bag containing an ocular therapeutic agent, for slow release delivery of the therapeutic agent and simultaneous support of a capsule of the extracted lens when implanted back in the patient's eye.

8. The device of claim 7 wherein the therapeutic agent is selected from the group consisting of steroids, non-steroidal anti-inflammatory drugs (NSAIDS), antibiotics, anti-fungals, antioxidants, macrolides including but not limited to cyclosporine, tacrolimis, rapamycin, mycophenolic acid and their analogs, voclosporin, immunomodulatores, etanercept, infliximab, adalimumab, antibodies, antiproliferative agents, gene delivery agents, neuroprotective agents, anti-glaucoma agents, and combinations thereof.

9. A method of treating an eye of a patient using the delivery body of claim 1 implanted in the eye.
